(19) United States
(12) Patent Application Publication
(10) Pub. No.: US 2009/0186774 A1
(43) Pub. Date: Jul. 23, 2009

(54) SEPSIS DETECTION MICROARRAY

(76) Inventors: Carrie Jane Turner, Wiltshire (GB); Amanda Marie Yates, Wiltshire (GB); Matthew Christopher Jackson, Salisbury (GB)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 12/301,724

(22) PCT Filed: May 16, 2007
(86) PCT No.: PCT/GB07/01772

§ 371 Date: Nov. 20, 2008

(30) Foreign Application Priority Data
May 20, 2006 (GB) 0610078.8

(51) Int. Cl. C40B 30/04; C40B 40/08; C40B 50/14; C12Q 1/68

(52) U.S. Cl. 506/9; 506/17; 506/30; 435/6

(57) ABSTRACT

The invention relates to the early detection of sepsis and the use of particular sets of biomarkers. Combinations of biomarkers representing changes in expression levels of specific genes are provided and, in particular, the use of microarrays to detect such changes of expression and to provide early diagnostic information is provided.

### BACKGROUND

Since its introduction in the mid-90s (Schera et al, 1995, Science 270: 368), DNA microarray technology has become widely used in many areas of the life sciences. It allows the rapid identification and quantification of specific nucleic acid sequences, for example mRNA indicative of gene expression. The basic principle is that of small scale solid-phase hybridisation of analytes to robotically printed, high-density arrays of immobilised nucleic acid probes, combined with automated processing, fluorescent detection, and sophisticated data acquisition and analysis software. This approach has led to the development of high-throughput analysis applicable to many areas of genomics and gene expression profiling. The use of microarrays comprising antibodies or antibody-like molecules, aptamers, phage and other ligands similarly allows analysis of a wide range of protein-protein and other cognate intermolecular interactions.

Since many pathological states are associated with characteristic changes in gene expression, often in specific sub-sets of cells, there have been a number of attempts to use microarray screening of biological samples as a diagnostic tool. An early example was the microarray analysis of inflammatory disease-related genes reported by Heller et al (1997, Proc Natl Acad Sci USA, 94: 2150). The basic principle is to examine changes in gene expression in target cells involved in the disease process in order to identify molecules that either have a direct involvement in the development of disease (and which might, therefore, represent potential targets for intervention) or earlier or more precise diagnostic markers.

One important condition where DNA microarray technology is being evaluated is that of the early detection of serious infections, and the identification of developing sepsis at as early a stage as possible. Despite greatly improved diagnosis, treatment and support, serious infection and sepsis remain significant causes of death and often result in chronic ill-health or disability in those who survive acute episodes. Although sudden, overwhelming infection is comparatively rare amongst otherwise healthy adults, it constitutes an increased risk in immunocompromised individuals, seriously ill patients in intensive care, burns patients and young children. In a proportion of cases, an apparently treatable infection leads to the development of sepsis; a dysregulated, inappropriate response to infection characterised by progressive circulatory collapse leading to renal and respiratory failure, abnormalities in coagulation, profound and unresponsive hypotension and, in about 30% of cases death. The incidence of sepsis in the population of North America is about 0.3% of the population annually (about 750,000 cases) with mortality rising to 40% in the elderly and to 50% in cases of the most severe form, septic shock (Angus et al, 2001, Crit Care Med 29: 1303-1310).

Following infection with infectious micro-organisms, the body reacts with a classical inflammatory response and activation firstly of the innate, non-specific immune response, followed by a specific, acquired immune response. In the case of bacterial infections, bacteraemia leads to the rapid (within 30-90 minutes) onset of pyrexia and release of inflammatory cytokines such as interleukin-1 (IL-1) and tumour necrosis factor-α (TNF-α) triggered by the detection of bacterial toxins, long before the development of a specific, antigen-driven immune response.

In Gram-negative bacteraemia due to infections such as typhoid, plague, tularaemia and brucellosis, or peritonitis from Gram-negative gut organisms such as Escherichia coli, Klebsiella, Proteus or Pseudomonas this is largely a response to lipopolysaccharide (LPS) and other components derived from bacterial cell walls. Circulating LPS and, in particular, its constituent lipid A, provokes a wide range of systemic reactions. It is probably contact with Kupffer cells in the liver that first leads to IL-1 release and the onset of pyrexia. Activation of circulating monocytes and macrophages leads to release of cytokines such as IL-6, IL-12, IL-15, IL-18, TNF-α, macrophage migration inhibitory factor (MIF), and cytokine-like molecules such as high mobility group B1 (HMGB1), which, in turn activate neutrophils, lymphocytes and vascular endothelium, up-regulate cell adhesion molecules, and induce prostaglandins, nitric oxide synthase and acute-phase proteins. Release of platelet activating factor (PAF), prostaglandins, leukotrienes and thromboxane activates vascular endothelium, regulates vascular tone and activates the extrinsic coagulation cascade. Dysregulation of these responses results in the complications of sepsis and septic shock in terms of peripheral vasodilation leading to hypotension, and abnormal clotting and fibrinolysis producing thrombosis and intravascular coagulation (Cohen, 2002, Nature 420: 885-891).

In the case of infection with Gram-positive pathogens, septic shock is associated with the production of exotoxins. For instance, toxic shock syndrome, a particularly acute form of septic shock that often affects otherwise healthy individuals is due to infection with particular strain of Staphylococcus aureus, which produces an exotoxin known as toxic shock syndrome toxin-1 (TSST-1). A similar syndrome is caused by invasive infection with certain group A Streptococcus pyogenes strains, and is often associated with streptococcal pyogenic enterotoxin A (SPE-A). Some Gram-positive exotoxins (including TSST-1) are thought to exert their effects predominantly as a result of their superantigen properties. Superantigens are able to non-specifically stimulate T lymphocytes by cross-linking MHC Class II molecules on antigen presenting cells to certain classes of T cell receptors. Usually, T cell receptor (TCR)-Major Histocompatibility Complex (MHC) interactions are highly specific, with only T cells carrying TCRs that specifically recognise short antigen-derived peptides presented by the MHC able to bind and be activated, ensuring an antigen-specific T cell response. Superantigens bypass this mechanism resulting in massive and inappropriate activation of T cells. However, SPE-A is not an efficient superantigen and some further mechanism must be implicated.

The ability to detect potentially serious infections as early as possible and, especially, to predict the onset of sepsis in susceptible individuals is clearly advantageous. A considerable effort has been expended over many years in attempts to establish clear criteria defining clinical entities such as shock, sepsis, septic shock, toxic shock and systemic inflammatory response syndrome (SIRS). Similarly, many attempts have been made to design robust predictive models based on measuring a range of clinical, chemical, biochemical, immunological and cytometic parameters and a number of scoring systems, of varying prognostic success and sophistication, proposed.

According to the 1991 Consensus Conference of the American College of Chest Physicians (ACCP) and Society of Critical Care Medicine (SCCM) “SIRS” is considered to be present when patients have more than one of the following: a body temperature of greater than 38° C. or less than 36° C., a heart rate of greater than 90/min, hyperventilation involving a respiratory rate higher than 20/min or PaCO₂ lower than 32 mm Hg, a white blood cell count of greater than 12000 cells/μl or less than 4000 cells/μl (Bone et al, 1992, Crit Care Med 20: 864-874).

“Sepsis” has been defined as SIRS caused by infection. It is accepted that SIRS can occur in the absence of infection in, for example, burns, pancreatitis and other disease states. “Infection” was defined as a pathological process caused by invasion of a normally sterile tissue, fluid or body cavity by pathogenic or potentially pathogenic micro-organisms.

“Severe sepsis” was defined as sepsis complicated by organ dysfunction, itself defined by Marshall et al (1995, Crit Care Med 23: 1638-1652) or the Sequential Organ Failure Assessment (SOFA) score (Ferreira et al, 2002, JAMA 286: 1754-1758).

“Septic shock” refers (in adults) to sepsis plus a state of acute circulatory failure characterised by a persistent arterial hypotension unexplained by other causes.

In order to evaluate the seriousness of sepsis in intensive care patients and to allow rational treatment planning, a large number of clinical severity models have been developed for sepsis, or adapted from more general models. The first generally accepted system was the Acute Physiology and Chronic Health Evaluation score (APACHE, and its refinements APACHE II and III) (Knaus et al, 1985, Crit Care Med 13: 818-829; Knaus et al, 1991, Chest 100: 1619-1636), with the Mortality Prediction Model (MPM) (Lemeshow et al, 1993, JAMA 270: 2957-2963) and the Simplified Acute Physiology (SAPS) score (Le Gall et al, 1984, Crit Care Med 12: 975-977) also being widely used general predictive models. For more severe conditions, including sepsis, more specialised models such as the Multiple Organ Dysfunction Score (MODS) (Marshall et al, 1995, Crit Care Med 23: 1638-1652), the Sequential Organ Failure Assessment (SOFA) score (Ferreira et al, 2002, JAMA 286: 1754-1758) and the Logistical Organ Dysfunction Score (LODS) (Le Gall et al, 1996, JAMA 276: 802-810) were developed. More recently, a specific model, PIRO (Levy et al, 2003, Intensive Care Med 29: 530-538), has been proposed. All of these models use a combination of a wide range of general and specific clinical measures to attempt to derive a useful score reflecting the seriousness of the patient's condition and likely outcome.

In addition to the standard predictive models described above, the correlation of sepsis and a number of specific serum markers has been extensively studied with a view to developing specific diagnostic and prognostic tests, amongst which are the following.

C-reactive protein (CRP) is a liver-derived serum acute phase protein that is well-known as non-specific marker of inflammation. More recently (Toh et al, 2003, Intensive Care Med 29: 55-61) a calcium dependent complex of CRP and very low density lipoprotein (VLDL), known as lipoprotein complexed C-reactive protein (LCCRP), has been shown to be involved in affecting the coagulation mechanism during sepsis. In particular, a common test known as the activated partial thromboplastin time develops a particular profile in cases of sepsis, and this has been proposed as the basis for a rapid diagnostic test.

TNF-α and IL-1 are archetypal acute inflammatory cytokines long known to be elevated in sepsis (Damas et al, 1989, Critical Care Med 17: 975-978) and have reported to be useful predictors of organ failure in adult respiratory distress syndrome, a serious complication of sepsis (Meduni et al, 1995, Chest 107: 1062-1073)

Activated complement product C3 (C3a) and IL-6 have been proposed as useful indicators of host response to microbial invasion, and superior to pyrexia and white blood cell counts (Groeneveld et a/, 2001, Clin Diagn Lab Immunol 8: 1189-1195). Secretory phospholipase A₂ was found to be a less reliable marker in the same study.

Procalcitonin is the propeptide precursor of calcitonin, serum concentrations of which are known to rise in response to LPS and to correlate with IL-6 and TNF-α levels. Its use as a predictor of sepsis has been evaluated (Al-Nawas et al, 1996, Eur J Med Res 1: 331-333). Using a threshold of 0.1 ng/ml, it correctly identified 39% of sepsis patients. However, other reports suggest that it is less reliable than the use of serial CRP measurements (Neely et al, 2004, J Burn Care Rehab 25: 76-80), although superior to IL-6 or IL-8 (Harbarth et al, Am J Resp Crit Care Med 164: 396-402).

Changes in neutrophil surface expression of leukocyte activation markers (such as CD11b, CD31, CD35, L-selectin, CD16) have been used as a marker of SIRS and have been found to correlate with IL-6 and subsequent development of organ failure (Rosenbloom et al, 1995, JAMA 274: 58-65). Similarly, expression of platelet surface antigens such as CD63, CD62P, CD36 and CD31 have been examined, but no reliable predictive model constructed.

Finally, it has been shown that down-regulation of monocyte HLA-DR expression is a predictor of a poor outcome in sepsis and may be an indication of monocyte deactivation, impairing TNF-α production. Treatment with IFN-γ has been shown to be beneficial in such cases (Docke et al, 1997, Nature Med 3: 678-681).

However, although many of these markers correlate with sepsis and some give an indication of the seriousness of the condition, no single marker or combination markers has yet been shown to be a reliable diagnostic test, much less a predictor of the development of sepsis. The 2001 International Sepsis Definition Conference concluded that “the use of biomarkers for diagnosing sepsis is premature” (Levy et al, 2003, Intensive Care Med 29: 530-538).

There have been previous attempts to apply DNA microarray technology to the analysis of infection and sepsis-related gene expression. Nau et al (US 2004/0038201) analysed genes activated in macrophages exposed to various pathogens using microarrays comprising probes representing a wide range of inflammation-related genes and identified nearly 200 as informative as biomarkers.

Similarly, Stuhlmuller et al (US 2005/0037344) disclose an extensive list of gene sequences said to be informative in microarray assays for monocyte/macrophage activation in response to pathogens.

Lu et al (US 2005/130185) disclose a sepsis detection microarray chip comprising a selection of at least 66 bacterially-derived sequences, which is used to analyse PCR-amplified purified DNA obtained from potentially infected blood samples. Kingsman et al (US 2005/0196817) disclose a microarray-based assay for sepsis based on a panel of known sepsis-related genes.

Extracting reliable diagnostic patterns and robust prognostic indications from changes over time in complex sets of variables including traditional clinical observations, clinical chemistry, biochemical, immunological and cytometric data requires sophisticated methods of analysis. The use of expert systems and artificial intelligence, including neural networks, for medical diagnostic applications has been being developed for some time (Place et al, 1995, Clinical Biochemistry 28: 373-389; Lisboa, 2002, Neural Networks 15: 11-39). Specific systems have been developed in attempts to predict survival of sepsis patients (Flanagan et al, 1996, Clinical Performance & Quality Health Care 4: 96-103) by use of multiple logistic regression and neural network models using APACHE scores and the 1991 ACCP/SCCM SIRS criteria described above (Bone et al, 1992, Crit Care Med 20: 864-874). Such studies suggest that, although both approaches can give good predictive results, neural network systems are less sensitive to preselected threshold values (results of a number of studies reviewed by Rosenberg, 2002, Curr Opin Crit Care 8:321-330). Brause et al (2004, Journal für Anästhesie und Intensivbehandlung 11: 40-43) provides an example of a neural network model being used for sepsis prediction. This model (MEDAN) analysed a range of standard clinical measure and compared its results with those obtained by using the APACHE II, SOFA, SAPS II, and MODS models. The study concluded that, of the markers available, the most informative were systolic and diastolic blood pressure, and platelet count.

Neural networks are non-linear functions that are capable of identifying patterns in complex data systems. This is achieved by using a number of mathematical functions that make it possible for the network to identify structure within a noisy data set. This is because data from a system may produce patterns based upon the relationships between the variables within the data. If a neural network sees sufficient examples of such data points during a period known as “training”, it is capable of “learning” this structure and then identifying these patterns in future data points or test data. In this way, neural networks are able to predict or classify future examples by modelling the patterns present within the data it has seen. The performance of the network is then assessed by its ability to correctly predict or classify test data, with high accuracy scores, indicating the network has successfully identified true patterns within the data. The parallel processing ability of neural networks is dependent on the architecture of its processing elements, which are arranged to interact according to the model of biological neurones. One or more inputs are regulated by the connection weights to change the stimulation level within the processing element. The output of the processing element is related to its activation level and this output may be non-linear or discontinuous. Training of a neural network therefore comprises an adjustment of interconnected weights depending on the transfer function of the elements, the details of the interconnected structure and the rules of learning that the system follows (Place et al, 1995, Clinical Biochemistry 28: 373-389). Such systems have been applied to a number of clinical situations, including health outcomes models of trauma patients (Marble & Healy (1999) Art Intell Med 15: 299-307).

Dybowski et al (1996, Lancet 347: 1146-1150) use Classification and Regression Trees (CART to select inputs from 157 possible sepsis prediction criteria and then use a neural network running a genetic algorithm to select the best combination of predictive markers. These include many routine clinical values and proxy indicators rather than serum or cell surface biomarkers. However, the problem being addressed is the prognosis of patients who already have a clear diagnosis of sepsis and are already critically ill.

A further refinement of the genetic algorithm approach involves the use of Artificial Immune Systems, of which one version is the Artificial Immune Recognition System (AIRS) (Timmis et al, An overview of Artificial Immune Systems. In: Paton, Bolouri, Holcombe, Parish and Tateson (eds.) “Computation in Cells and Tissues: Perspectives and Tools for Thought”, Natural Computation Series, pp 51-86, Springer, 2004; Timmis (L. N. De Castro and J, Timmis. Artificial Immune Systems: A New Computational Intelligence Approach. Springer-Verlag, 2002). which are adaptive systems inspired by the clonal selection and affinity maturation processes of biological immune systems as applied to artificial intelligence.

Immunologically speaking, AIRS is inspired by the clonal selection theory of the immune system (F. Burnett. The Clonal Selection Theory of Acquired Immunity. Cambridge University Press, 1959). The clonal selection theory attempts to explain that how, through a process of matching, cloning, mutation and selection, antibodies are created that are capable of identifying infectious agents. AIRS capitalises on this process, and through a process of matching, cloning and mutation, evolves a set of memory detectors that are capable of being used as classifiers for unseen data items. Unlike other immune inspired approaches, such as negative selection, AIRS is specifically designed for use in classification, more specifically one-shot supervised learning.

US patent application 2002/0052557 describes a method of predicting the onset of a number of catastrophic illnesses based on the variability of the heart-rate of the patient. Again, a neural network is among the possible methods of modelling and analysing the data.

International patent application WO 00/52472 describes a rapid assay method for use in small children based on the serum or neutrophil surface levels of CD11b or ‘CD11b complex’ (Mac-1, CR3). The method uses only a single marker, and one which is, arguably, a well-known marker of neutrophil activation in response to inflammation.

International application WO2004/043236 discloses certain informative combinations of biomarkers for predicting the onset of sepsis. Using amplified cDNA to probe an expression microarray for informative biomarkers at −48 h, −24 h and time of onset of clinical sepsis, a different set of biomarkers was selected for each time point by a process known as multiple additive regression trees (MART). At −48 h, the most informative biomarkers were found to be CD4, ARG2 (arginase type II), MMP9 (matrix metallopeptidase 9), HSPA1A (Hsp70) and LBP (lipopolysaccharide-binding protein). At −24 h, the selected biomarkers were FCGR1A (CD64, immunoglobulin Fcγ receptor type IA), ARG2, CD4, IL-8, TLR4 (toll-like receptor 4) and CSF2 (colony-stimulating factor 2). The corresponding set at the time of onset of sepsis was FCGR1A, ARG2, CD86 (B7-2), IL18R1 (IL-18 receptor 1), MMP9, CD4, IL-1β, IL-8 and IL-4.

The ability to detect the earliest signs of infection and/or sepsis has clear benefits in terms of allowing treatment as soon as possible. Indications of the severity of the condition and likely outcome if untreated inform decisions about treatment options. This is relevant both in vulnerable hospital populations, such as those in intensive care, or who are burned or immunocompromised, and in other groups in which there is an increased risk of serious infection and subsequent sepsis. The use or suspected use of biological weapons in both battlefield and civilian settings is an example where a rapid and reliable means of testing for the earliest signs of infection in individuals exposed would be advantageous.

Despite the greater knowledge of both the molecular basis of, and physiological response to, sepsis a need remains for a method of predicting sepsis as early as possible in the course of an infection, preferably during the therapeutic window of intervention, prior to the onset of clinical symptoms and disease. It is an object of the invention to identify novel markers and combinations of biomarkers, preferably useful for screening by means of micro-array technology. The approach of the prior art described above may be characterised as the selection of genes known to be in some way related to the processes of the inflammatory or immunological response to infection and testing their usefulness in microarrays or other types of assay. This is logical but presupposes that the processes involved in the earliest stages of infection are well-characterised and that the earliest genes to be activated are known. It also fails to consider the possibility of informative epiphenomena, that is, genes that are activated incidentally, or as part of a parallel or peripheral response. An alternative approach is to screen a wide range of potentially expressed (and, in some cases, apparently completely unconnected) gene sequences to identify those which, despite this, are nevertheless useful predictors of infection, either alone or in combination.

CD40 is a TNF-receptor superfamily member expressed on T and B lymphocytes, among other cells, and is required for a wide variety of immune and inflammatory responses, in particular B cell immunoglobulin production and isotype switching, and development of memory B cells (Grewel & Flavell, 1998, Annu Rev Immunol 16: 111). Its ligand is another leukocyte cell surface molecule, CD154. Two alternately spliced isoforms are known, the longer isoform (1) being encoded by transcript variant 1 (NCBI accession number NM 001250, SEQ ID NO:1).

CD5 is also a cell surface receptor expressed on T and B lymphocytes where it interacts with its ligand CD72 and has a role in modulating the immune response (Berland & Wortis, 2002, 20: 253). The cDNA sequence encoding human CD5 has the NCBI accession number NM 014207 (SEQ ID NO:2).

CD79A, previously known as MB-1 or Ig-α, is part of the B cell antigen receptor complex together with another similar molecule, CD79B (B29 or Ig-β), and the surface immunoglobulin chains. CD79A and B are involved in signal transduction and B cell surface immunoglobulin expression Jumaa et al, 2005, Annu Rev Immunol 23: 415). There two known transcript variants, and the longer transcript sequence is listed at NCBI accession number NM 001783 (SEQ ID NO:3).

CRX is the gene for cone-rod homeobox, a homeodomain transcription factor that controls differentiation in photoreceptor cells and is required for normal cone and rod cell function. Mutations in this gene are associated with photoreceptor degeneration (Leber congenital amaurosis type III and autosomal dominant cone-rod dystrophy 2, but no immunological functions are known (Chen et al, 2002, Human Molecular Genetics, 11: 873). The cDNA sequence is available at NM 000554 (SEQ ID NO:4).

CTNND1 is the gene encoding catenin (cadherin-associated protein) delta-1, a member of the armadillo family of proteins (previously known as p120 cas and p120 catenin). It is one of a number of proteins (others being β-catenin and plakaglobin) that bind to the cytoplasmic region of cadherins, modulating cell adhesion and linking cadherins to the cytoskeleton (Franze & Ridley, 2004, J Biol Chem 279: 6588). Such molecules may also have a role in signal transduction through rho family GTPases. The cDNA sequence is available at NM 001331 (SEQ ID NO:5).

CX3CL1 encodes chemokine (C-X3-C motif) ligand 1, an unusual chemokine (previously known as fractalkine) characterised by the unique spacing of the first 2 cysteines in its chemokine cysteine motif and its dual role as a chemoattractant and cell adhesion molecule involved in the inflammatory response. It is expressed as a cell surface molecule but a soluble from is generated by juxtamembrane proteolytic cleavage (Umehara et al, 2004, Arterioscler Thromb Vasc Biol 24: 34). The cDNA sequence is available at NM 002996 (SEQ ID NO:6).

ENTPD2 is the gene for ectonucleoside triphosphate diphosphohydrolase 2 (otherwise known as CD39L or NTPDase-2). ENTPD5 is the related ectonucleoside triphosphate diphosphohydrolase 5 (CD39L4 or NTPDase-5). These molecules are cell surface ATP-hydrolyzing enzymes responsible for the breakdown of extracellular nucleotides, thus regulating a complex system of cell signalling via large families of purine and pyrimidine receptors. ENTPD2 exists in a number of splice variants, which may have distinct functions (Wang et al, 2005, Biochem J 385: 729). A long isoform is encoded by the cDNA sequence of NM 203468 (SEQ ID NO:7). NM 001246 encodes a shorter isoform with a truncated C-terminus. The ENTPD5 sequence is available at NM 001249 (SEQ ID NO:8).

EPHA8 is a gene encoding the ephrin A8 receptor, a member of the ephrin receptor subfamily of receptor tyrosine kinases. The ephrin A8 receptor functions as a receptor for ephrin A2, A3 and A5 and is involved in short-range contact-mediated axonal guidance during development of the nervous system (Gu et al, 2005, Oncogene 24: 4243). There is a splice variant shortened at the C-terminus (not yet detected at the protein level) but the longer isoform is encoded by the sequence of NM 020526 (SEQ ID NO:9)

GPR44 encodes G protein-coupled receptor 44, more widely known as chemoattractant receptor-homologous molecule expressed on Th2 cells (CRTH2). This the prostaglandin D₂ (PGD₂) receptor responsible for mediating the inflammatory effect of PGD₂ on a variety of leukocytes and other cells (Hata et al, 2005, J Biol Chem 280: 32442). It is implicated in the skewing of the T cell response to a Th2 pattern during sepsis and low levels of expression of CRTH2 are associated with a poor outcome (Venet et al, 2004, Clin Immunol 113: 278). The sequence is available at NM 004778 (SEQ ID NO:10).

HDAC5 is histone deacetylase 5, a class II histone deacetylase that represses transcription when tethered to a promoter. Histone acetylation/deacetylation alters chromatin structure and is a major factor controlling gene expression. HDAC5 is thought to interact with MEF2 family proteins and may play a role in myogenesis (Zhang et al, 2002, Mol Cell Biol 22: 7302). There are two known isoforms encoded by two splice variants. NM 001015053 relates to the longer transcript (SEQ ID NO:11).

HMMR is the gene for hyaluronan-mediated motility receptor (RHAMM). RHAMM is thought to be involved in invasion and metastasis of tumour cells. Although widely expressed on tumour cells, in normal tissue its expression is limited to testis, placenta and thymus. There is a truncated splice variant lacking an internal segment. NM 012484 represents the longest transcript (SEQ ID NO:12).

IL-8 is very widely known as a member of the CXC family of chemokines and is a prime mediator of the inflammatory response, being a potent chemotactic and angiogenic factor. It has been reported to be a relatively poor predictor of sepsis (Harbarth et al, Am J Resp Crit Care Med 164: 396). The sequence is available at NM 000584 (SEQ ID NO:13).

MAP1A encodes microtubule-associated protein 1A, a member of a family of microtubule-associated proteins involved in microtubule assembly. MAP1A is expressed predominantly in the brain. The functional protein comprises light and heavy chains resulting from proteolytic processing of a single propeptide encoded by the sequence of NM 002373 (SEQ ID NO:14).

MAPK7 is the gene encoding mitogen-activated protein kinase 7 (MAP kinase 7 or ERK5). The MAP kinases occupy a central role in the intracellular signalling cascades from a number of receptor tyrosine kinases and G protein-coupled receptors but MAPK7 differs from the others in that it has not only protein kinase activity but also is also capable of translocating to the nucleus where it appears to be able to phosphorylate and activate transcription factors directly (Buschbeck & Ullrich, 2005, J Biol Chem 280: 2659). Four alternative transcripts encoding two distinct isoforms have been reported. The longest transcript is represented by the sequence of NM 002749 (SEQ ID NO:15).

MEF2D is the gene for MADS box transcription enhancer factor 2, polypeptide D (myocyte enhancer factor 2D). Originally described as a muscle-specific transcription factor, MEF2 is now known to exist as four alternatively spliced isoforms (A-D) that are differentially expressed in a range of tissues (Zhu et al, J Biol Chem, 2005, 280: 28749). MEF2D appears to be involved in leukocyte activation and chromosomal translocations resulting in MEF2D fusion proteins contribute to the development of some acute lymphoblastic leukaemias (Prima et al, 2005, Leukemia 19: 806). The MEF2D sequence is available as NM 005920 (SEQ ID NO:16).

ODF1 is outer dense fibre of sperm tails 1 and encodes the major protein of the outer dense fibre layer surrounding the axoneme of sperm tails. Defects in the outer dense fibres lead to abnormal sperm morphology and infertility. There is no known connection with genes involved with the inflammatory response. The sequence is available as NM 022410 (SEQ ID NO: 17).

SAA3P denotes the serum amyloid A3 pseudogene. The serum amyloid A (SAA) superfamily consists of two acute phase genes, SAA1 and SAA2 and a constitutively expressed gene, SAA4.

SAA3P appears to be non-expressed pseudogene. The predicted open reading frame contains an insertion causing a frameshift, which generates a premature stop codon. The resultant hypothetical protein has been expressed. The genomic sequence is available as NG 002634 (SEQ ID NO: 18).

SLC6A9 is solute carrier family 6 (neurotransmitter transporter, glycine) member 6 (GLYT1). A member of a large superfamily of transporter proteins, SLC6A9 is a sodium:glycine symporter, which may be involved in inhibitory glycinergic neurotransmission. There are a number of splice variants encoding three known isoforms. The longest transcript (giving rise to isoform 2) is available as NM 201649 (SEQ ID NO:19).

SPN is the gene for CD43 (leukosialin, sialophorin). Leukosialin is a major sialoglycoprotein of most leukocytes. It appears to play a part in modulating cell-cell interactions, including T cell activation (Daniels et al, 2002, Nature Immunol 3: 903). The cDNA sequence is available at NM 003114 (SEQ ID NO: 20).

TDGF1 is teratocarcinoma-derived growth factor 1 (previously known as Cripto). It is a cell surface, glycosyl phosphatidylinositol (GPI)-anchored molecule, a member of the EGF-CFC family of growth factor-like molecules (Shen, 2003, J Clin Invest 112: 500). It is over-expressed in a wide range of carcinomas but is not known to have a role in inflammation or the immune response. The cDNA sequence is at NM 003212 (SEQ ID NO: 21).

TSC22D1 is TSC22 domain family member 1. It is the founding member of the TSC22 family of early response gene transcription factors and is particularly involved in the TGF-β signalling pathway (and was formerly known as TGF-β1-induced transcript 4—TGFB1I4) (Gupta et al, 2003, J Biol Chem 278: 7331). The accession number is NM 006022 (SEQ ID NO: 22).

It is an object of the invention to provide a microarray-based method of diagnosing and predicting serious infection and sepsis.

### STATEMENT OF INVENTION

The invention discloses a DNA microarray and a method for its use in the screening of biological samples for the early detection of infection and/or sepsis. The invention discloses a set of up to 22 sequences, which when used in combination, are shown to be highly predictive of sepsis, despite the fact that many of them are not related to genes known or expected to be associated with the response to infection.

In a first aspect, the invention provides a microarray detection chip for the detection of infection in a patient, comprising a plurality of nucleic acid probes immobilised on a solid substrate, at least 7 of said probes comprising a nucleic acid sequence derived from a gene in the list consisting of CD40 (SEQ ID NO:1), CD5 (SEQ ID NO:2), CD79A (SEQ ID NO:3), CRX (SEQ ID NO:4), CTNND1 (SEQ ID NO:5), CX3CL1 (SEQ ID NO:6), ENTPD2 (SEQ ID NO:7), ENTPD5 (SEQ ID NO:8), EPHA8 (SEQ ID NO:9), GPR44 (SEQ ID NO:10), HDAC5 (SEQ ID NO:11), HMMR (SEQ ID NO:12), IL-8 (SEQ ID NO:13), MAP1A (SEQ ID NO:14), MAPK7 (SEQ ID NO:15), MEF2D (SEQ ID NO:16), ODF1 (SEQ ID NO:17), SAA3P (SEQ ID NO:18), SLC6A9 (SEQ ID NO:19), SPN (SEQ ID NO:20), TDGF1 (SEQ ID NO:21) and TSC22D1 (SEQ ID NO:22). More preferably, the microarray comprises at least 8 such probes, most preferably 10 such probes.

Reference to a nucleic acid sequence in the context of binding or hybridisation includes its complementary sequence, as will be understood by those of skill in the art.

In the case of SAA3P (SEQ ID NO: 18) the probe has 100% homology with the serum amyloid A3 pseudogene. However, the serum amyloid A (SAA) superfamily also contains at least two similar acute phase genes, SAA1 and SAA1, which are expressed in response to acute inflammation. The probe has a 48/50 match with the human serum amyloid A (GSAA1 gene, GenBank accession number X13895, SEQ ID NO: 45).

Preferably each probe comprises at least 10 contiguous nucleotides and more preferably each sequence is from an open reading frame. More preferably the probe comprises at least 25, further preferably 40 and most preferably 50 such contiguous nucleotides. In one favoured embodiment the probes are synthetic oligonucleotides, preferably DNA, more preferably single-stranded DNA.

In a highly preferred embodiment each probe comprises contiguous nucleotides from one of the oligonucleotide sequences depicted in Table 1 (SEQ ID NO: 23-44).

In another aspect the invention provides a method of detecting infection comprising the steps of:
a. extracting mRNA from a sample from a patient
b. amplifying said mRNA by an in vitro transcription reaction
c. hybridising the product of the in vitro transcription reaction with the immobilised probes on the microarray chip of any preceding claim
d. detecting in vitro transcription products bound to one or more probes
e. analysing the pattern of binding in order to assess the likelihood of infection

Preferably the hybridisation is performed under conditions of sufficient stringency as to minimise non-specific binding whilst allowing specific binding of complementary nucleic acid sequences. More preferably it is performed at between 40 and 44° C., most preferably at 42° C. Selection of hybridisation and washing conditions are within ordinary skill of practitioners in the field. Further details are available in standard texts such as Bowtell & Sambrook, DNA Microarrays: A Molecular Cloning Manual (Cold Spring Harbor Laboratory Press, Cold Spring Harbor, N.Y., 2002).

As an alternative to amplifying and/or reverse transcribing extracted mRNA, such mRNA may be detected by direct hybridisation and binding to the probes immobilised on the microarray.

It is preferred that analysis of the pattern of binding comprises use of a neural network.

In a final aspect, the invention provides a method of manufacturing a microarray detection chip comprising the steps of;
a. synthesising at least 10 nucleic acid probes each comprising at least 10 contiguous nucleotides from a nucleic acid sequence selected from SEQ ID NOs 1-22.
b. applying each probe to a solid substrate in predetermined array.

Alternatively, each probe comprises at least 10 contiguous nucleotides from a nucleic acid sequence selected from SEQ ID NOs 23-44.

TABLE 1
Genes and corresponding probes of the invention

| | | | | |
|---|---|---|---|---|
| Gene | NCBI Acc. No. | SEQ ID | 50-mer probe | SEQ ID |
| name | (cDNA*) | NO | | NO |

| | | | | |
|---|---|---|---|---|
| CD40 | NM 001250 | 1 | TTCCTTGCGG TGAAAGGGAA TTCCTAGACA | 23 |
| | | | CCTGGAACAG AGAGACACAC | |
| CD5 | NM 014207 | 2 | CGCCTGTCAG CTTATCCAGC TCTGGAAGGG | 24 |
| | | | GTTCTGCATC GCTCCTCCAT | |
| CD79A | NM 001783 | 3 | CAGGAGGGCA ACGAGTCATA CCAGCAGTCC | 25 |
| | | | TGCGGCACCT ACCTCCGCGT | |
| CRX | NM 000554 | 4 | CCGTCTCTGA CCTCCGCCCC CTATGCCATG | 26 |
| | | | ACCTACGCCC CGGCCTCCGC | |
| CTNND1 | NM 001331 | 5 | CCAGCGTAGT ATGGGCTATG ATGACCTGGA | 27 |
| | | | TTATGGTATG ATGTCTGATT | |
| CX3CL1 | NM 002996 | 6 | CGCTACATC CCCCGGAGCT GTGGTAGTAA | 28 |
| | | | TTCATATGTC CTGGTGCCCGT | |
| ENTPD2 | NM 203468 | 7 | TCTCTGCCTT CTTCTACACT GTGGACTTTT | 29 |
| | | | TGCGGACTTC GATGGGGCTG | |
| ENTPD5 | NM 001249 | 8 | AAGTGTGTGA TAACTTGGAA AACTTCACCT | 30 |
| | | | CAGGCAGTCC TTTCCTGTGC | |
| EPHA8 | NM 020526 | 9 | CTTCGCTGCG GGCGGATACT CCTCTCTGGG | 31 |
| | | | CATGGTGCTA CGCATGAACG | |
| GPR44 | NM 004778 | 10 | CTGGGCACCA CCTTCTGCAA ACTGCACTCC | 32 |
| | | | TCCATCTTCT TTCTCAACAT | |
| HDAC5 | NM 001015053 | 11 | ACGAGTTCTC ACCTGATGTG GTCCTAGTCT | 33 |
| | | | CCGCCGGGTT TGATGCTGTT | |
| HMMR | NM 012484 | 12 | TTTGCCCTGA AGACCCCATT AAAAGAAGGC | 34 |
| | | | AATACAAACT GTTACCGAGC | |
| IL8 | NM 000584 | 13 | CAGTGCATAA AGACATACTC CAAACCTTTC | 35 |
| | | | CACCCCAAAT TTATCAAAGA | |
| MAP1A | NM 002373 | 14 | TAAGCTCATG CCACACATGA AGAATGAACC | 36 |
| | | | CACTACTCCC TCATGGCTGG | |
| MAPK7 | NM 002749 | 15 | TGGCTCCAGC ACCCCAGGAG TTTTGCCTTA | 37 |
| | | | CTTCCCACCT GGCCTGCCGC | |
| MEF2D | NM 005920 | 16 | TTCCATGCCC ACTGCCTACA ACACAGATTA | 38 |
| | | | CCAGTTGACC AGTGCAGAGC | |
| ODF1 | NM 024410 | 17 | CCCTGCAACC CGTGCAGCCC ATATGATCCT | 39 |
| | | | TGCAACCCGT GTTATCCCTG | |
| SAA3P* | NG 002634 | 18 | TCTCCCAGAG TCCTCCCTTG GAAAGCAGAG | 40 |
| | | | AATGGGAAGG TGGGCTGTTG | |
| SLC6A9 | NM 201649 | 19 | GCCTACAACT CTGGTCTCCT TCCCCAACTC | 41 |
| | | | ATGGCCCAGC ACTCCCTAGC | |
| SPN | NM 003123 | 20 | CTAGCCGTCG GCCCACGCTC ACCACTTTCT | 42 |
| | | | TTGGCAGACG GAAGTCTCGC | |
| TDGF1 | NM 003212 | 21 | AACTGGGATT AGTTGCCGGG CTGGGCCATC | 43 |
| | | | AGGAATTTGC TCGTCCATCT | |
| TSC22D1 | NM 006022 | 22 | AACGCTTCTG TGAGACTTGA TAATAGCTCC | 44 |
| | | | TCTGGTGCAA GTGTGGTAGC | |

*SAA3P is thought to be a non-expressed pseudogene, NG 002634 is derived from a genomic sequence.

### DETAILED DESCRIPTION OF THE INVENTION

The invention will be described in further detail with reference to the following Example.

### Example 1

### Microarray Design and Fabrication

A custom human immune response array was designed homologous to the DSTL-designed murine immune function array with additional genes that had been identified from the previous sepsis study. A total of 1438 genes were represented by a single 50-mer oligonucleotide designed by MWG Biotech. In addition the array contained 768 oligonucleotides from the MWG Biotech commercially available ‘diverse function’ genes to act as an inter-microarray slide control. Printing of the oligonucleotides was performed by MWG according to their array layout plan with the entire set of printed spots (2206) triplicated on each slide.

### Blood Samples for Analysis

Blood samples were taken from intensive care unit (ICU) patients and mixed with blood/bone marrow RNA stabilisation reagent (Roche) in a 1:10 ratio as per the manufacturer's instructions. Stabilised samples were shipped to DSTL frozen (−20° C.) and subsequently stored at −70° C. prior to mRNA extraction.

### RNA Isolation

Messenger RNA (mRNA) was isolated from 27.5 mls blood lysate (corresponding to 2.5 mls of stabilised blood) using the mRNA Isolation Kit for Blood/Bone Marrow (Roche) following the manufacturers guidelines with a few minor changes (volumes for the 55 ml lysate protocol were halved, centrifugation was for 3 minutes, washing of MGP beads was performed using 1 ml MGP washing buffer repeated 3 times and elution was into 20 μl of redistilled water). The entire mRNA preparation was treated with RNase free DNase from the DNA-free kit (Ambion Inc.) following the manufacturers guidelines. The final mRNA preparation was quantitated by A₂₆₀.

mRNA Amplification and Fluorescent Dye Labelling

All amplification and labelling steps were performed with the Amino Allyl MessageAmp™ aRNA kit (Ambion Inc.) following the manufacturers instructions. Cy3 and Cy5 post-labelling reactive dyes used in the protocol were obtained from Amersham Bioscience. Amplification of mRNA was performed using 50 ng purified mRNA. A total of 3 μg of amplified mRNA was fluorescently labelled for hybridisation, 1.5 μg with Cy3 and 1.5 μg with Cy5. Following labelling, the same sample labelled with either Cy3 or Cy5 were mixed together and purified using the MessageAmp™ kit. The volume of eluted sample was reduced to 9 μl by drying in a vacuum drier. Following this, the size of the labelled amplified mRNA was reduced for hybridisation using the Fragmentation kit (Ambion Inc.)

### Microarray Hybridisation

Microarray slides were prepared for hybridisation by attaching a GeneFrame® (MWG) over the oligo printed area according to the manufacturers instructions. Fragmented, labelled mRNA (11 μl) was denatured for 3 minutes at 95° C., snap-cooled on ice for 3 minutes and briefly centrifuged. 240 μl MWG hybridisation solution was added to the sample and mixed before applying to the microarray slide. The slide was covered with a plastic coverslip which attaches to the GeneFrame® and placed within a HC2 hybridisation cassette (CamLab). 500% water was added to each well of the cassette to prevent drying. The closed cassette was placed in a 42° C. hybridisation oven for 16 hours. After hybridisation, slides were removed from the cassettes and the GeneFrame® and coverslip removed. Slides were washed sequentially using three buffers (1×SSC, 0.2% SDS; 0.5×SSC and 0.25×SSC). Each wash was for 5 minutes with agitation. Slides were centrifuged for 5 minutes at 1500 rpm and dried slides stored in the dark until scanning. Slides were scanned using a GenePix 4000B microarray scanner (Axon Instruments Inc.). PMT voltages for 635 and 532 nm channels were adjusted to yield a total pixel intensity ratio of approximately 1:1. Images were saved as single image TIFF files.

### Microarray Gene Expression Analysis

TIFF files from the Axon scanners were loaded into BlueFuse software (BlueGnome Ltd) and processed to ‘fused’ data following the manufacturers instructions. The resultant data files were saved and subsequently analysed in GeneSpring software.

### Neural Network

For analysis, data was collated from patients 1 to 6 days prior to the onset of sepsis and compared with a control group consisting of ICU patients who did not develop sepsis. Individual samples provided data measuring up to 22 different parameters and selective combinations of variables were fed into a multi-layered perceptron neural network (Proforma, Hanon Solutions, Glasgow, Scotland).

Each network was trained with a random 70% selection of balanced sepsis and control data using back propagation algorithms and then tested with the remaining 30% of the data. This process was then repeated, using a different 70% of randomised data, until a total of 5 repeats had been run.

The predictive abilities of these 5 models were then averaged to give an overall predictive capability of the network. The most successful network was the one most capable of correctly classifying previously unseen patients as being from either the sepsis or non-sepsis control group.

### Results

Table 2 shows various sets of genes selected from the 22 most informative genes based on their individual scores. The sets were assigned in such a way as to attempt establish the relative importance of combinations of genes based on such factors as their individual scores (sets B and G representing the top and bottom ranked genes of the 22), whether or not genes with known immunological or inflammatory functions were included (set E with CD40 and IL-8 excluded, for instance) and the effect of larger or smaller sets.

TABLE 2

| A | B | C | D | E | F | G | H | I | J |
|---|---|---|---|---|---|---|---|---|---|
| MEF2 | MEF2D | CD40 | IL-8 | MEF2D | SAAP3 | TDGF1 | SPN | SPN | HMMR |
| ENTPD2 | ENTPD2 | ENTPD2 | SAAP3 | ENTPD2 | MAP1A | HDAC5 | CD40 | EPHA8 | SAAP3 |
| CD40 | CD40 | EPHA8 | CD40 | EPHA8 | EPHA8 | ENTPD5 | SAAP3 | CD40 | ENTPD2 |
| EPHA8 | EPHA8 | IL-8 | SPN | SAA3P | CD40 | ODF1 | IL-8 | CRX | MAP1A |
| SAA3P | SAA3P | SAAP3 | MEF2D | HMMR | IL-8 | GPR44 | MEF2D | TSC22D1 | MAPK7 |
| HMMR | HMMR | MEF2D | EPHA8 | MAPK7 | CRX | CD5 | EPHA8 | MAPK7 | TSC22D |
| IL8 | IL8 | SPN | MAPK7 | SPN | SPN | CD79A | MAP1A | HMMR | IL-8 |
| MAPK7 | MAPK7 | | ENTPD2 | TSC22D1 | MEF2D | CTNND1 | CRX | MEF2D | |
| SPN | SPN | | HMMR | | MAPK7 | CX3CL1 | | ENTPD2 | |
| TSC22D1 | TSC22D1 | | | | HMMR | SLC6A9 | | | |
| MAP1A | | | | | ENTPD2 | | | | |
| CRX | | | | | TSC22D1 | | | | |
| SLC6A9 | | | | | | | | | |
| TDGF1 | | | | | | | | | |
| HDAC5 | | | | | | | | | |
| CTNND1 | | | | | | | | | |
| ENTPD5 | | | | | | | | | |
| CD79A | | | | | | | | | |
| CD5 | | | | | | | | | |
| GPR4 | | | | | | | | | |
| ODF1 | | | | | | | | | |
| CX3CL1 | | | | | | | | | |

Table 3 shows the ranked scores obtained following the neural network analysis

TABLE 3

| | | % correct | % correct | % correct |
|---|---|---|---|---|
| Rank | Gene set | Overall | Sepsis | Control |
| 1 | F | 97.5 | 100 | 90 |
| 2 | H | 87.3 | 83.3 | 94.7 |
| 3 | B | 86.3 | 86.5 | 85.7 |
| 4 | I | 84.4 | 92.6 | 72.2 |
| 5 | J | 87.3 | 94.2 | 75 |
| 6 | G | 87.2 | 95 | 60 |
| 7= | D | 83.7 | 92.9 | 66.7 |
| 7= | E | 83.7 | 92.9 | 66.7 |
| 9 | C | 79.5 | 82.6 | 75 |
| 10 | A | 70 | 69.6 | 71.4 |

Surprisingly, set B, comprising the top ten-scoring genes based on their individual scores did not give the best overall predictive value. Even more surprisingly, the best predictive set, set F, comprised set B together with two genes not known to have any connection with the immune or inflammatory response, CRX and MAP1A. Overall, the values indicate that the inclusion of genes that could not have been predicted to be useful based on their known functions nevertheless resulted in improved predictive scores.

1. A microarray detection chip for the detection of infection in a patient comprising at least 7 nucleic acid probes immobilized immobilised on a solid substrate, said probes each comprising at least 10 contiguous nucleotides from a nucleic acid sequence of a gene selected from the group consisting of CD40, CD5, CD79A, CRX, CTNND1, CX3CL1, ENTPD2, ENTPD5, EPHA8, GPR44, HMMR, IL-8, MAP1A, MAPK7, MEF2D, ODF1, SAA3P, SLC6A9, SPN, TDGF1, TSC22D1 and HDAC5.

2. The microarray detection chip of claim 1, wherein each probe comprises at least 10 contiguous nucleotides from a nucleic acid sequence selected from SEQ ID NOs: 1-22.

3. The microarray detection chip of claim 1, wherein each probe comprises at least 10 contiguous nucleotides from a nucleic acid sequence selected from SEQ ID NOs: 23-44.

4. The microarray detection chip of claim 1, wherein the probes each comprise at least 25 contiguous nucleotides from said nucleic acid sequence.

5. The microarray detection chip of claim 1, wherein the probes each comprise at least 40 contiguous nucleotides from said nucleic acid sequence.

6. The microarray detection chip of claim 1, comprising at least 8 of said nucleic acid probes.

7. A method of detecting infection, SIRS, or sepsis comprising the steps of:
a. extracting mRNA from a sample from a patient;
b. amplifying said nucleic acid by in vitro transcription reaction;
c. hybridizing the product of the in vitro transcription reaction with the immobilized probes on the microarray detection chip of claim 1;
d. detecting the in vitro transcription reaction product bound to one or more probes; and
e. analyzing the pattern of binding in order to assess the likelihood of infection, SIRS or sepsis.

8. The method of claim 7, wherein the analysis step (e) comprises use of a neural network.

9. A method of manufacturing a microarray detection chip comprising the steps of:
a. synthesizing at least 10 nucleic acid probes, wherein each nucleic acid probe comprises at least 10 contiguous nucleotides from a nucleic acid sequence selected from SEQ ID NOs: 1-22; and
b. applying each probe to a solid substrate in a predetermined array.

10. A method of manufacturing a microarray detection chip comprising the steps of;
a. synthesizing at least 10 nucleic acid probes, wherein each nucleic acid probe comprises at least 10 contiguous nucleotides from a nucleic acid sequence selected from SEQ ID NOs 23-44; and
b. applying each probe to a solid substrate in a predetermined array.

11. A method for detecting an infection, SIRS, or sepsis in a patient, comprising:
a. extracting a nucleic acid from a sample from the patient;
b. optionally amplifying said nucleic acid by in vitro transcription reaction;
c. hybridizing either the nucleic acid or the product of the in vitro transcription reaction with a combination of one or more probes, wherein each probe is derived from a nucleic acid sequence of a different gene selected from the group consisting of CD40, CD5, CD79A, CRX, CTNND1, CX3CL1, ENTPD2, ENTPD5, EPHA8, GPR44, HMMR, IL-8, MAP1A, MAPK7, MEF2D, ODF1, SAA3P, SLC6A9, SPN, TDGF1, TSC22D1, and HDAC5; and
d. detecting binding of the nucleic acid or in vitro transcription reaction product to the one or more probes.

12. The method of claim 11, wherein said combination comprises at least seven probes.

13. The method of claim 11, wherein each probe comprises at least 10 contiguous nucleotides of the nucleic acid sequence of the gene selected from the group consisting of CD40, CD5, CD79A, CRX, CTNND1, CX3CL1, ENTPD2, ENTPD5, EPHA8, GPR44, HMMR, IL-8, MAP1A, MAPK7, MEF2D, ODF1, SAA3P, SLC6A9, SPN, TDGF1, TSC22D1, and HDAC5.

14. A method for detecting an infection, SIRS, or sepsis in a patient, comprising:
a. extracting a nucleic acid from a sample from the patient;
b. optionally amplifying said nucleic acid by in vitro transcription reaction;
c. hybridizing either the nucleic acid or the product of the in vitro transcription reaction with a combination of one or more probes, wherein each probe is derived from a nucleic acid sequence of a different oligonucleotide sequence of SEQ ID NO:23 or SEQ ID NO: 24; and
d. detecting binding of the nucleic acid or in vitro transcription reaction product to the one or more probes.

15. The method of claim 14, wherein said combination comprises at least seven probes.

16. The method of claim 14, wherein each probe comprises at least 10 contiguous nucleotides of SEQ ID NO:23 or SEQ ID NO:24.
